(19) United States
(12) Patent Application Publication
(10) Pub. No.: US 2020/0337540 A1
(43) Pub. Date: Oct. 29, 2020

(54) ENDOSCOPE SYSTEM

(71) Applicant: OLYMPUS CORPORATION, Tokyo (JP)

(72) Inventor: Satoshi TAKEKOSHI, Tokyo (JP)

(73) Assignee: OLYMPUS CORPORATION, Tokyo (JP)

(21) Appl. No.: 16/864,828

(22) Filed: May 1, 2020

Related U.S. Application Data
(63) Continuation of application No. PCT/JP2018/032442, filed on Aug. 31, 2018.

(30) Foreign Application Priority Data
Nov. 6, 2017 (JP) 2017-214019

(51) Int. Cl. A61B 1/06; A61B 1/05; A61B 1/00; A61B 1/04

(52) U.S. Cl. CPC A61B 1/0638; A61B 1/05; A61B 1/00009; A61B 1/043; A61B 1/00188

(57) ABSTRACT

An endoscope system is provided with: an illumination device configured to alternately irradiate first and second light with different wavelengths by time division; an imaging lens capable of changing a focus position; an imaging sensor configured to acquire each of first and second images related to the first and second light; and a processor. The processor detects a first focused position based on the first image and detects a second focused position based on the second image; and the processor drives the imaging lens to the first focused position prior to acquiring the first image, and drives the imaging lens to the second focused position prior to acquiring the second image.

### CROSS REFERENCE TO RELATED APPLICATION

This application is a continuation application of PCT/JP2018/032442 filed on Aug. 31, 2018 and claims benefit of Japanese Application No. 2017-214019 filed in Japan on Nov. 6, 2017, the entire contents of which are incorporated herein by this reference.

### BACKGROUND OF THE INVENTION

### 1. Field of the Invention

The present invention relates to an endoscope system that forms a plurality of optical images from a plurality of lights with different wavelengths to acquire a plurality of image signals.

### 2. Description of the Related Art

In a medical field, fluorescent observation has been conventionally performed in which, for example, a fluorescent agent is administered to a subject, and excitation light is irradiated to the subject to diagnose whether there is a lesion site or not, and the like based on an emission state of fluorescence from the subject. More specifically, in a surgical operation, blood flow assessment of tissue, lymph node identification and the like are performed by performing infrared fluorescence observation using, for example, ICG (indocyanine green).

As an example of performing such fluorescent observation, an endoscope system described in WP2016/072172 is given, the endoscope system including: a first filter in which a transmittance of a first wavelength band including an excitation wavelength for causing fluorescence to occur is a predetermined transmittance, and a transmittance of a second wavelength band is a first transmittance; a second filter in which the transmittance of the first wavelength band is the predetermined transmittance, and the transmittance of the second wavelength band is a second transmittance; a light source device configured to emit lights of the first and second wavelength bands by interposing the first or second filter on an optical path of light emitted from a light source; a camera unit; and a controlling portion configured to perform, after performing control for maintaining brightness of a reflect light image at a reference value, control for switching a filter to be interposed on the optical path from the first filter to the second filter when the brightness of the reflect light image is below the reference value.

Further, not only in the medical field but also in fields other than the medical field, for example, an industrial endoscope field, it is also possible to irradiate visible light and special light (for example, infrared light and ultraviolet light) that is different from the visible light in wavelength to acquire a visible light image and acquire a special light image.

It is known that, at the time of forming an image of a plurality of lights with different wavelengths, axial chromatic aberration occurs on an optical system.

### SUMMARY OF THE INVENTION

An endoscope system according to one aspect of the present invention is provided with: an illumination device configured to alternately irradiate first light and second light that is different from the first light in wavelength to a subject by time division; an imaging lens configured to form an image of light from the subject as an optical image, a focus position of the imaging lens being changeable; an imaging sensor configured to pick up the optical image of the subject to which the first light is irradiated by the illumination device to acquire a first image signal and pick up the optical image of the subject to which the second light is irradiated by the illumination device to acquire a second image signal; and a processor. The processor detects a first focused position using the first image signal without using the second image signal and detects a second focused position using the second image signal without using the first image signal; and the processor drives the imaging lens to the first focused position prior to acquiring the first image signal, and drives the imaging lens to the second focused position prior to acquiring the second image signal.

An endoscope system according to one aspect of the present invention is provided with: an illumination device configured to irradiate first light and second light that is different from the first light in wavelength to a subject; an imaging lens configured to form an image of light from the subject as an optical image, a focus position of the imaging lens being fixed; an imaging sensor configured to pick up the optical image of the subject to which the first light is irradiated by the illumination device to acquire a first image signal and pick up the optical image of the subject to which the second light is irradiated by the illumination device to acquire a second image signal; and a processor. The processor detects an amount of blur around an area shown by the second image signal; and the processor performs gradation conversion processing for reducing gradations of the second image signal so that the number of gradations after performing the gradation conversion processing becomes smaller as the detected amount of blur becomes larger.

### BRIEF DESCRIPTION OF THE DRAWINGS

FIG. 1 is a block diagram showing a configuration of an endoscope system in a first embodiment of the present invention;

FIG. 2 is a chart showing spectral transmission characteristics of an excitation light cut filter in the first embodiment;

FIG. 3 is a timing chart showing a state of time-division acquisition of a white light image and a fluorescent image in the endoscope system of the first embodiment;

FIG. 4 is a chart showing an example of focus detection for the white light image and an example of focus detection for the fluorescent image in a focus detecting portion of the first embodiment;

FIG. 5 is a flowchart showing operation of the endoscope system in the first embodiment;

FIG. 6 is a block diagram showing a configuration of an endoscope system in a second embodiment of the present invention;

FIG. 7 is a flowchart showing fluorescent image processing in the endoscope system in the second embodiment;

FIG. 8 is a chart showing an example of spatial distribution of luminances of a fluorescent part before gradation conversion processing in the second embodiment;

FIG. 9 is a chart showing an example of spatial distribution of luminances of the fluorescent part after the gradation conversion processing of a fluorescent image is performed, in the second embodiment;

FIG. 10 is a chart showing an example of displaying a blurred area around a fluorescent area in color different from color of the fluorescent area in the second embodiment; and

FIG. 11 is a table showing an example of causing the number of gradations after performing the gradation conversion processing according to an amount of blur of the fluorescent image to change, in the second embodiment.

### DETAILED DESCRIPTION OF THE PREFERRED EMBODIMENTS

Embodiments of the present invention will be described below with reference to drawings.

### First Embodiment

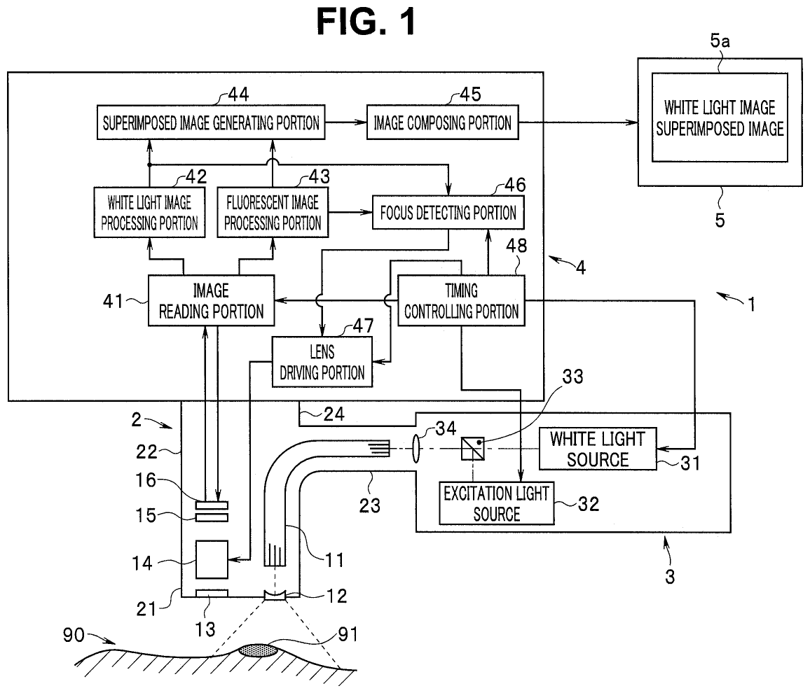

FIGS. 1 to 5 show a first embodiment of the present invention, and FIG. 1 is a block diagram showing a configuration of an endoscope system 1.

For example, as shown in FIG. 1, the endoscope system 1 is provided with an endoscope 2, a light source portion 3, an image operating portion 4 and a monitor 5.

The endoscope 2 is an imaging sensor configured to be inserted into a body cavity of a subject 90 to pick up an image of living tissue or the like in the body cavity and output an image signal.

The light source portion 3 is a light source device configured to supply light to be irradiated to the subject 90, to the endoscope 2.

The image operating portion 4 is a processor configured to generate and output an observation image by performing various image processing for the image signal outputted from the endoscope 2.

The monitor 5 is a display device configured to display the observation image outputted from the image operating portion 4 on a screen 5a.

The endoscope 2 is provided with an insertion portion 21 formed in an elongated shape insertable into the body cavity of the subject 90, an operation portion 22 provided on a proximal end side of the insertion portion 21, a light guide cable 23 provided, for example, extending from the operation portion 22 and being attachable to and detachable from the light source portion 3, and a signal cable 24 provided extending from the operation portion 22 and being attachable to and detachable from the image operating portion 4.

Here, the operation portion 22 is configured in a shape that can be grasped by a user such as a surgeon and is provided with a scope switch (not shown) configured with one or more switches capable of outputting various instruction signals according to operations by the user to the image operating portion 4.

A light guide 11 for transmitting light supplied from the light source portion 3 is inserted in an inside of the light guide cable 23 and a distal end portion of the insertion portion 21.

The light guide 11 is configured, for example, as a light guide fiber bundle which is a plurality of optical fibers bundled, and an incident end of the light guide 11 is arranged on an optical path of light supplied from the light source portion 3 via a condensing lens 34. On an optical path of light transmitted by the light guide 11 and emitted from an emission end of the light guide 11, an illumination lens 12 is disposed. Thus, light is irradiated to the subject 90 by the illumination lens 12.

An illuminating portion (an illumination device) configured to irradiate first light and second light that is different from the first light in wavelength to the subject 90 by time division in the present embodiment is configured having the light source portion 3 including the condensing lens 34, the light guide 11 and the illumination lens 12. As described later, the first light is normal light (white light, reference light or the like) for acquiring return light from the subject 90 in a specific example of the present embodiment. The second light is excitation light for exciting a fluorescent agent administered to the subject 90 to acquire fluorescence from the fluorescent agent in the specific example of the present embodiment.

On the distal end portion of the insertion portion 21, the illumination lens 12 described above is arranged, and an objective window 13 for causing light from the subject 90 to be incident is arranged.

On an optical path of the light caused to be incident from the objective window 13, an imaging lens 14 and an image pickup device 16 are provided.

The imaging lens 14 forms an image of light from the subject 90 as an optical image, and a focus position is changeable.

The image pickup device 16 is configured to perform photoelectric conversion of the optical image formed by the imaging lens 14 to generate an electric image signal.

Furthermore, at any position on an optical path from the objective window 13 to the image pickup device 16 (between the imaging lens 14 and the image pickup device 16 in the example shown in FIG. 1), an excitation light cut filter 15 is arranged.

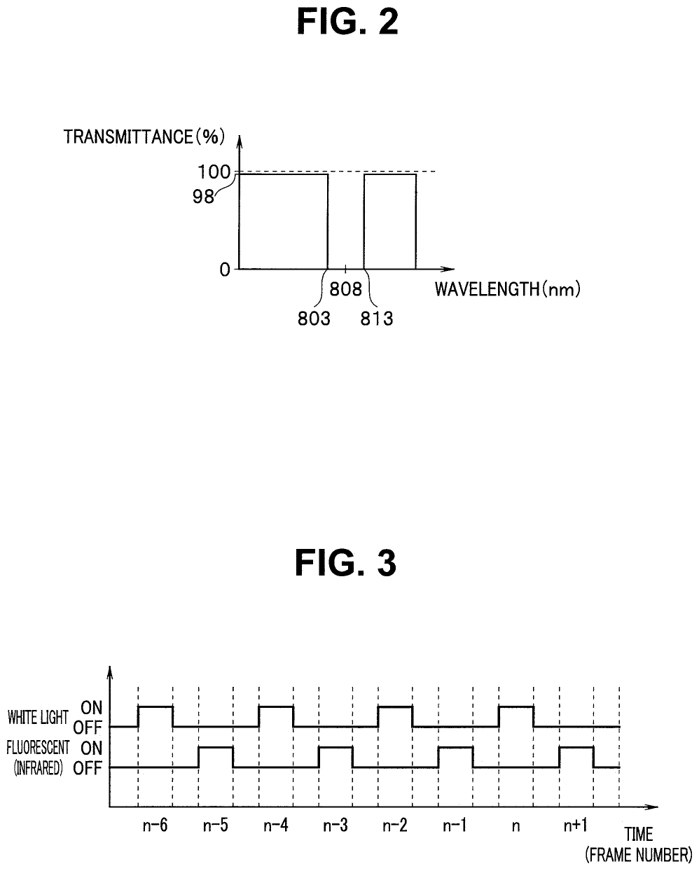

Here, FIG. 2 is a chart showing spectral transmission characteristics of the excitation light cut filter 15.

In the present embodiment, it is assumed that ICG (indocyanine green) is used as a fluorescent agent (however, the fluorescent agent is not limited to ICG), and an excitation wavelength is 808 nm. Here, from ICG to which excitation light with the wavelength of 808 nm is irradiated, fluorescence, which is near infrared light belonging to a wavelength band on a side with a longer wavelength than the wavelength 808 nm of the excitation light, is emitted.

Hereinafter, description will be made on a case where ICG is used as a fluorescent agent, and narrow-band light including light with the wavelength of 808 nm (to be near infrared light) is used as excitation light unless otherwise stated. Further, in the present embodiment, it is assumed that, as normal light for acquiring return light from the subject 90, white light (however, the normal light is not limited to white light, and reference light, for example, G light or B light is also possible) is used.

Therefore, as schematically shown in FIG. 2, the excitation light cut filter 15 is provided with such spectral transmission characteristics that the narrow-band light including the light with the wavelength of 808 nm, for example, light of a band of 803 to 813 nm is cut (a transmittance is caused to be almost 0%), and light with other wavelengths are transmitted with a high transmittance (for example, 98%).

The image pickup device 16 is a color image pickup device, for example, with a primary color filter (though a complementary color filter is also possible, description will be made below on an assumption that the color filter is a primary color filter) attached to an image pickup surface, and, specifically, a color CMOS image sensor is assumed. The image pickup device 16 is configured to perform an image pickup operation according to an image pickup device driving signal outputted from an image reading portion 41 of the image operating portion 4, which is to be described later, and output an acquired image signal to the image reading portion 41.

An image pickup portion (an imaging sensor) in the present embodiment configured to pick up an optical image (an optical image formed by the imaging lens 14) of the subject 90 to which first light (normal light) is irradiated by the illuminating portion (the light source portion 3, the light guide 11 and the illumination lens 12) to acquire a first image signal (a normal image signal) and pickup an optical image (an optical image formed by the imaging lens 14) of the subject 90 to which second light (excitation light) is irradiated by the illuminating portion to acquire a second image signal (a fluorescent image signal) is configured having the excitation light cut filter 15 and the image pickup device 16.

Next, the light source portion 3 is provided, for example, with a white light source 31, an excitation light source 32, a beam splitter 33 and the condensing lens 34.

The white light source 31 is configured being provided with, for example, a white LED (or an R-LED, a G-LED and a B-LED) or a xenon (Xe) lamp and is capable of emitting white light (the first light) as normal light.

The excitation light source 32 is a light source configured to emit the narrow-band light including the light with the wavelength of 808 nm as excitation light (the second light that is different from the first light in wavelength), and, for example, a laser diode (LD) that emits the light with the wavelength of 808 nm is used.

The white light source 31 and the excitation light source 32 are controlled by a timing controlling portion 48 to emit light by time division as described later.

The beam splitter 33 is provided, for example, with a dichroic mirror surface configured to reflect the light with the wavelength of 808 nm, which is excitation light emitted from the excitation light source 32, and light with a wavelength equal to or longer than 808 nm with a reflectance of almost 100%, and transmit white light emitted from the white light source 31 with a transmittance of almost 100%.

The condensing lens 34 condenses the white light or the excitation light emitted from the beam splitter 33 to the incident end of the light guide 11.

The image operating portion 4 is configured having the image reading portion 41, a white light image processing portion 42, a fluorescent image processing portion 43, a superimposed image generating portion 44, an image composing portion 45, a focus detecting portion 46, a lens driving portion 47 and the timing controlling portion 48. Note that each portion of the image operating portion 4 may be configured as an individual electronic circuit or may be configured as a circuit block in an integrated circuit such as an FPGA (field programmable gate array).

The image reading portion 41 transmits an image pickup device driving signal to the image pickup device 16 to cause the image pickup device 16 to perform an image pickup operation as described above based on a control signal from the timing controlling portion 48 and receives an image signal outputted from the image pickup device 16 by the image pickup operation.

Furthermore, if the control signal from the timing controlling portion 48 is a control signal showing acquisition of a white light image, the image reading portion 41 transmits the image signal inputted from the image pickup device 16 to the white light image processing portion 42. If the control signal from the timing controlling portion 48 is a control signal showing acquisition of a fluorescent image, the image reading portion 41 transmits the image signal inputted from the image pickup device 16 to the fluorescent image processing portion 43. Thus, the image reading portion 41 is adapted to function as a selector configured to change an output destination of an inputted image signal based on a control signal from the timing controlling portion 48.

The white light image processing portion 42 is an image processing portion configured to perform various kinds of image processing, for example, demosaicing, white balance correction, noise removal and gamma correction for a white light image signal, which is the first image signal inputted from the image reading portion 41 and is a normal image signal. Note that when the normal image signal is a reference light image signal, a reference light image processing portion is arranged instead of the white light image processing portion 42.

The fluorescent image processing portion 43 is an image processing portion configured to perform various kinds of image processing, for example, demosaicing, noise removal, conversion to a luminance signal and gamma correction for a fluorescent image signal, which is the second image signal inputted from the image reading portion 41, to generate a fluorescent image signal configured with a luminance signal.

The superimposed image generating portion 44 converts the fluorescent image configured with the luminance signal, which is inputted from the fluorescent image processing portion 43, to a signal of a display color (though G is given here as an example of a color that can be visually easily identified because a color component of red is relatively large in a living body, the color is not limited to G but may be an arbitrary color obtained by arbitrarily setting a ratio of RGB (R: red; G: green; B: blue). Then, the superimposed image generating portion 44 superimposes the fluorescent image converted to the display color and the white light image inputted from the white light image processing portion 42 after adjusting a signal level as necessary to generate a superimposed image.

Then, the superimposed image generating portion 44 outputs the fluorescent image converted to the display color, the white color image inputted from the white light image processing portion 42 and the generated superimposed image to the image composing portion 45.

The image composing portion 45 outputs one of the fluorescent image, the white light image and the superimposed image that have been inputted from the superimposed image generating portion 44 to the monitor 5 as a composite image, or the image composing portion 45 combines two or more to generate a composite image and outputs the generated composite image to the monitor 5.

Here, a form of the image outputted from the image composing portion 45 is decided based on a setting made by the user if the setting is made, and decided based on a predetermined initial setting if the setting by the user is not made. Here, the setting by the user can be made by an operation panel or a switch not shown, which is provided on the image operating portion 4, or the scope switch or the like of the endoscope 2.

As some examples of the composite image generated by the image composing portion 45, a parallel display image configured with the fluorescent image and the superimposed image, a parallel display image configured with the white light image and the superimposed image, a parallel display image configured with the fluorescent image and the white light image, and a parallel display image configured with three of the fluorescent image, the white light image and the superimposed image are given. At the time of performing parallel display, display sizes may be caused to differ according to images, or it is also possible to provide a display area for another image in a part of a display area of a certain image. Further, though parallel display is given as an example here, an aspect of displaying any of the image combinations on the monitor 5 by time division is also possible.

Based on a control signal from the timing controlling portion 48, the focus detecting portion 46 detects a first focused position using a white light image signal which is a first image signal acquired in the past and inputted from the white light image processing portion 42 (without using a fluorescent image signal acquired in the past) when it is a white light image that is to be acquired next and detects a second focused position using a fluorescent image signal which is a second image signal acquired in the past and inputted from the fluorescent image processing portion 43 (without using a white light image signal acquired in the past) when it is a fluorescent image that is to be acquired next.

Here, the focus detection by the focus detecting portion 46 assumes contrast AF in which a plurality of images with different focus positions are acquired by performing so-called wobbling for causing a focus position to change, and contrast values of the respective acquired images are detected to cause a focus position that corresponds to a contrast-value peak position to be a focused position. Then, the focus detecting portion 46 outputs the detected focused position to the lens driving portion 47.

The lens driving portion 47 is configured to, based on a control signal from the timing controlling portion 48, drive the imaging lens 14 to the first focused position prior to acquiring a white light image signal which is the first image signal and drive the imaging lens 14 to the second focused position prior to acquiring a fluorescent image signal which is the second image signal.

The timing controlling portion 48 transmits a control signal to each of the portions in the image operating portion 4 including the image reading portion 41, the focus detecting portion 46 and the lens driving portion 47 and each of the portions in the light source portion 3 including the white light source 31 and the excitation light source 32 to perform control to acquire a white light image signal and a fluorescent image signal by time division.

The monitor 5 is a display device capable of color display and displays an image outputted from the image composing portion 45 on the screen 5a.

Next, an operation and the like of the endoscope system 1 of the present embodiment will be described. Note that, hereinafter, description will be made on an assumption that, before fluorescent observation is performed, ICG is administered to the subject 90 as a fluorescent agent in advance, and a fluorescent part 91 (see FIG. 1 and the like) where the fluorescent agent exists in the subject 90. Further, though an operation in a white light observation mode for irradiating white light to observe only a white light image is also possible in the endoscope system 1 of the present embodiment, description about the white light observation mode will be omitted, and an operation and the like in a fluorescent observation mode will be described.

First, by operating, for example, a fluorescent observation start switch (not shown) of the image operating portion 4 after connecting each portion of the endoscope system 1 and turning on power, the user inputs an instruction signal to start fluorescent observation of an object (a fluorescent observation start signal) to the endoscope system 1.

Then, the user inserts the insertion portion 21 into the body cavity of the subject 90 and arranges the distal end portion of the insertion portion 21 near to the fluorescent part 91 which is an observation target.

When detecting the fluorescent observation start signal, the timing controlling portion 48 generates a control signal for causing a timing of occurrence of white light by the white light source 31, a timing of occurrence of excitation light by the excitation light source 32, and operations of the respective portions in the endoscope system 1 including an image pickup operation of the image pickup device 16 controlled by the image reading portion 41, a focus detection operation in the focus detecting portion 46 and a focus operation of the imaging lens 14 in the lens driving portion 47 to be synchronized, and outputs the control signal to each portion.

More specifically, if a next exposure period is a period during which a white light image is exposed, the timing controlling portion 48 generates a control signal for detecting a first focused position using a white light image signal without using a fluorescent image signal and outputs the control signal to the focus detecting portion 46. Further, if the next exposure period is a period during which a fluorescent image is exposed, the timing controlling portion 48 generates a control signal for detecting a second focused position using a fluorescent image signal without using a white light image signal and outputs the control signal to the focus detecting portion 46.

Furthermore, the timing controlling portion 48 outputs a control signal for driving the imaging lens 14 based on a focused position detected by the focus detecting portion 46, to the lens driving portion 47. Thereby, the lens driving portion 47 drives the imaging lens 14 to the first focused position prior to acquiring a white light image signal and drives the imaging lens 14 to the second focused position prior to acquiring a fluorescent image signal.

Further, for example, during a blanking period during which exposure is not performed, the timing controlling portion 48 generates a control signal for causing white light and excitation light to occur alternately (by time division) and outputs the control signal to the white light source 31 and the excitation light source 32 alternately. Thereby, during an exposure period, one of the white light source 31 and the excitation light source 32 emits light (the white light source 31 and the excitation light source 32 alternately emit light for each exposure period).

Then, the timing controlling portion 48 generates a control signal related to an image pickup operation and outputs the control signal to the image reading portion 41. Then, the image reading portion 41 transmits an image pickup device driving signal for causing an image pickup operation, for example, in a rolling shutter method to be performed, to the image pickup device 16 to cause the image pickup device 16 to perform the image pickup operation.

Thereby, during a white light emission period, white light emitted by the white light source 31 is irradiated to the object; an image of reflected light of the white light, which is return light from the object, is picked up by the image pickup device 16; and a white light image signal is outputted from the image pickup device 16.

During an excitation light emission period, excitation light emitted by the excitation light source 32 is irradiated to the object; excitation light in return light from the object is cut by the excitation light cut filter 15; only fluorescence is transmitted through the excitation light cut filter 15; and an image of the fluorescence is formed on the image pickup device 16 and picked up; and a fluorescent image signal is outputted from the image pickup device 16.

Furthermore, the timing controlling portion 48 generates a control signal, for example, for setting an output destination of an image signal inputted to the image reading portion 41 during the white light emission period to the white light image processing portion 42 and setting an output destination of an image signal inputted to the image reading portion 41 during the excitation light emission period to the fluorescent image processing portion 43 and outputs the control signal to the image reading portion 41. Thereby, an image signal obtained by performing exposure during the white light emission period is image-processed by the white light image processing portion 42; an image signal obtained by performing exposure during the excitation light emission period is image-processed by the fluorescent image processing portion 43; and each of the image signals is outputted to the superimposed image generating portion 44.

The superimposed image generating portion 44 superimposes a white light image signal (a normal image signal) and a fluorescent image signal acquired adjoining each other in order of time division as described above to generate a superimposed image. Thereby, such a superimposed image that the fluorescent part 91 is shown, for example, in green on a white light image is generated, and the superimposed image is outputted to the image composing portion 45.

As described above, the image composing portion 45 generates a composite image for displaying one or more of the fluorescent image, the white light image and the superimposed image in a predetermined display aspect based on an initial setting or a display aspect set by the user, and outputs the composite image to the monitor 5. Thereby, the composite image is displayed on the screen 5a of the monitor 5.

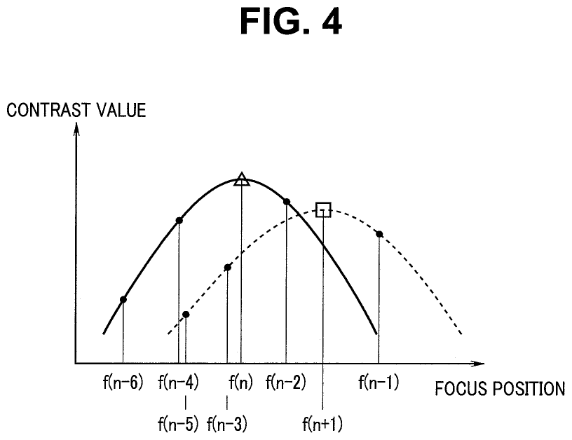

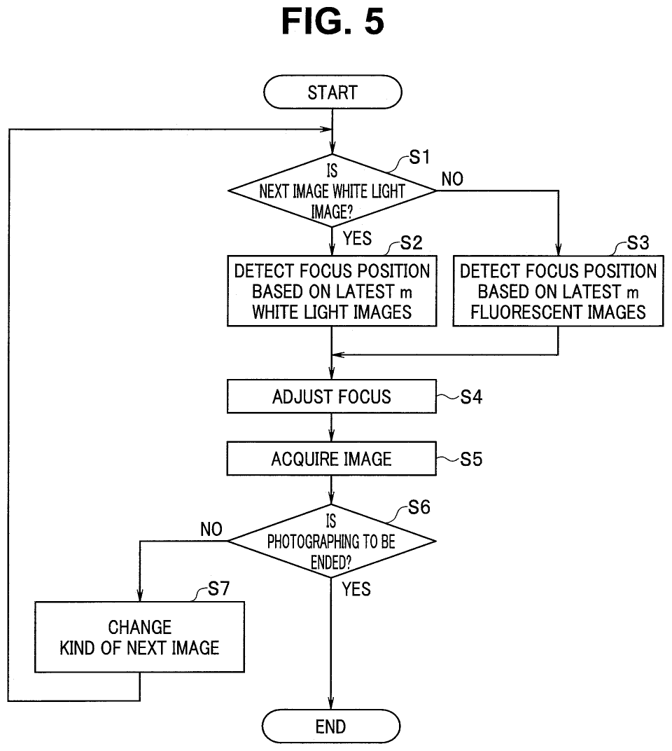

Next, an operation related to focus adjustment in the endoscope system 1 will be described along FIG. 5 with reference to FIGS. 3 and 4.

First, FIG. 3 is a timing chart showing a state of time-division acquisition of a white light image and a fluorescent image in the endoscope system 1.

As shown in FIG. 3, the timing controlling portion 48 controls each portion in the endoscope system 1 so that white light images and fluorescent images are alternately acquired by time division. In FIG. 3, it is shown that a white light image is exposed and acquired when white light is on, and a fluorescent image is exposed and acquired when fluorescence (infrared) is on (when the white light is off, exposure and acquisition of a white light image is not performed, and when fluorescence (infrared) is off, exposure and acquisition of a fluorescent image is not performed.)

Under such control related to an image pickup operation, an operation related to focus adjustment is performed as shown in FIG. 5. Here, FIG. 5 is a flowchart showing operation of the endoscope system 1.

When the process is started, the focus detecting portion 46 judges whether an image to be acquired next (more specifically, an image of a next frame) is a white light image or not based on a control signal from the timing controlling portion 48 (step S1).

If judging that the image to be acquired next is a white light image at step S1, the focus detecting portion 46 detects a focus position based on latest m (m is an integer equal to or larger than 1 and is preferably an integer equal to or larger than 3) white light images (step S2).

On the other hand, if judging that the image to be acquired next is not a white light image (that is, the image is a fluorescent image) at step S1, the focus detecting portion 46 detects a focus position based on the latest m fluorescent images (step S3).

Here, FIG. 4 is a chart showing an example of focus detection for a white light image (see a solid curved line) and an example of focus detection for a fluorescent image (see a dotted curved line) in the focus detecting portion 46. FIG. 4 shows an example in a case where m=3 is set.

A case where the white light image to be acquired next at step S2 is an image of an n-th frame in FIG. 3 will be given as an example. At this time, the latest three white light images are images acquired at (n−6)th, (n−4)th and (n−2)th frames.

Then, at step S2, the focus detecting portion 46 estimates a contrast-value peak position based on a focus position f(n−6) and a contrast value (assumed to be C(n−6) though not shown; the same hereinafter) of the image of the (n−6)th frame, a focus position f(n−4) and a contrast value C(n−4) of the image of the (n−4)th frame, and a focus position f(n−2) and a contrast value C(n−2) of the image of the (n−2)th frame, and sets a focus position f(n) corresponding to the estimated peak position as a focused position of the white light image to be acquired next.

Similarly, a case where the fluorescent image to be acquired next at step S3 is an image of an (n+1)th frame in FIG. 3 will be given as an example. At this time, the latest three fluorescent images are images acquired at (n−5)th, (n−3)th and (n−1)th frames.

Then, at step S3, the focus detecting portion 46 estimates a contrast-value peak position based on a focus position f(n−5) and a contrast value (assumed to be C(n−5) though not shown; the same hereinafter) of the image of the (n−5)th frame, a focus position f(n−3) and a contrast value C(n−3) of the image of the (n−3)th frame and a focus position f(n−1) and a contrast value C(n−1) of the image of the (n−1)th frame, and sets a focus position f(n+1) corresponding to the estimated peak position as a focused position of the fluorescent image to be acquired next.

Thus, the focus detecting portion 46 is adapted to detect a first focused position for a white light image to be acquired next using past white light image signals (without using past fluorescent image signals) and detect a second focused position for a fluorescent image to be acquired next using past fluorescent image signals (without using past white light image signals).

Note that though the example in which m=3 is set has been given here, m≥4 may be set to increase accuracy of detecting the contrast-value peak position, or m=1 or m=2 may be set if a focused position can be estimated from contrast values of one or two frames by other techniques.

When the processing of step S2 or step S3 has been performed, the lens driving portion 47 drives the imaging lens 14 to the focus position acquired from the focus detecting portion 46 based on a control signal from the timing controlling portion 48 and performs focus adjustment (step S4).

Then, the image pickup device 16 picking up an optical image formed by the imaging lens 14 at the adjusted focus position, and an image signal is acquired (step S5).

After that, the timing controlling portion 48 judges whether or not an instruction to end image pickup has been inputted from a switch or the like (step S6).

Here, if judging that the instruction to end image pickup has not been inputted yet, the timing controlling portion 48 changes a kind of a next image (that is, sets fluorescent image as the kind of the image to be acquired next if a white light image signal has been acquired at step S5 and sets white light image as the kind of the image to be acquired next if a fluorescent image signal has been acquired at step S5) (step S7) and returns to step Si to repeat the operation as described above.

On the other hand, if judging at step S6 that the instruction to end image pickup has been inputted, the timing controlling portion 48 ends the process.

Note that though fluorescent observation has been given as an example of irradiating first light and second light that is different from the first light in wavelength by time division to perform observation in the above description, the present invention is not limited to the case but can be widely applied to a case where observation is performed with a plurality of kinds of lights with different wavelengths such as narrow-band light observation (NBI: narrow band imaging).

Though an example of a medical endoscope system has been mainly described in the above description, the present invention is not limited to a medical endoscope system but can be applied to endoscope systems for fields other than the medical field, for example, an industrial endoscope system.

According to the first embodiment as described above, in the endoscope system 1 that irradiates first light and second light that are different in wavelength by time division, to detect a first focused position and drive the imaging lens 14 using a first image signal acquired by irradiating the first light (without using a second image signal) is performed prior to acquiring the first image signal, and to detect a second focused position and drive the imaging lens 14 using a second image signal acquired by irradiating the second light (without using the first image signal) is performed prior to acquiring the second image signal. Therefore, it is possible to reduce sharpness difference (or difference in image blur) that occurs on an image because an image forming position differs as a light wavelength differs. Thereby, it becomes possible to suppress an uncomfortable feeling at the time of observing an image according to the first image signal and an image according to the second image signal.

Further, when the first light is assumed to be normal light (white light, reference light or the like), and the second light is assumed to be excitation light for acquiring fluorescence from a fluorescent agent, it is possible to construct the endoscope system 1 that is appropriate for fluorescent observation.

Since the superimposed image generating portion 44 is further provided, a normal image signal and a fluorescent image signal are simultaneously observed on a superimposed image. Therefore, the sharpness difference between images is more remarkable, and the uncomfortable feeling is clearly felt. However, such an uncomfortable feeling can be appropriately reduced by the configuration described above.

### Second Embodiment

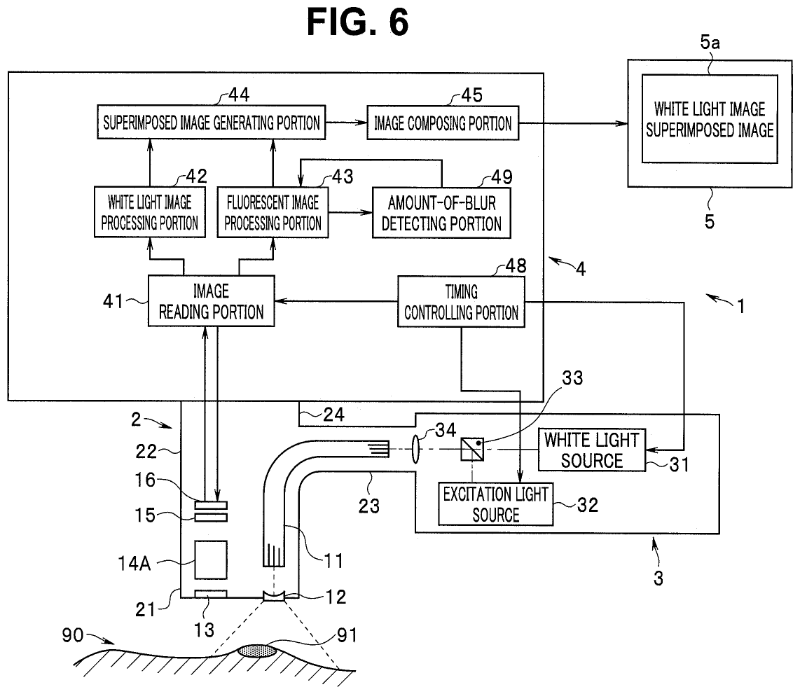

FIGS. 6 to 11 show a second embodiment of the present invention, and FIG. 6 is a block diagram showing a configuration of an endoscope system 1.

In the second embodiment, portions similar to portions of the first embodiment described above will be given the same reference numerals, and description of the portions will be appropriately omitted. Only different points will be mainly described.

Though the focus position of the imaging lens 14 of the first embodiment is configured to be changeable, the focus position of an imaging lens 14A of the present embodiment is configured to be fixed, and the focus position cannot be automatically changed for each frame. In the present embodiment, image processing (electric processing) is performed for at least one of a first image signal and a second image signal to reduce sharpness difference between images of the first image signal and the second image signal.

In other words, the endoscope 2 of the present embodiment is provided with the fixed-focus imaging lens 14A. The image operating portion 4 is not provided with the lens driving portion 47 of the first embodiment described above but is provided with an amount-of-blur detecting portion 49 instead of the focus detecting portion 46 (however, since the contrast value detected by the focus detecting portion 46 can be used to detect an amount of blur as described later, the focus detecting portion 46 may be used as the amount-of-blur detecting portion 49). The amount-of-blur detecting portion 49 is configured to detect an amount of blur around a fluorescent area shown by a fluorescent image signal.

In the present embodiment, it is assumed that, when the user makes an adjustment so that a white light image is focused, by manually adjusting an axial-direction position of the insertion portion 21 of the endoscope 2, a fluorescent image is in a state of being somewhat blurred due to axial chromatic aberration. Therefore, by the amount-of-blur detecting portion 49 detecting an amount of blur based on a fluorescent image inputted to the fluorescent image processing portion 43 and outputting the detected amount of blur to the fluorescent image processing portion 43, the fluorescent image processing portion 43 performs image processing to cause sharpness of the fluorescent image to be close to sharpness of a white light image (more specifically, gradation conversion processing for reducing gradations) and outputs a white light image, which is a processing result, to the superimposed image generating portion 44.

Thus, the fluorescent image processing portion 43 is an image processing portion configured to perform the gradation conversion processing for reducing gradations of a fluorescent image which is the second image signal.

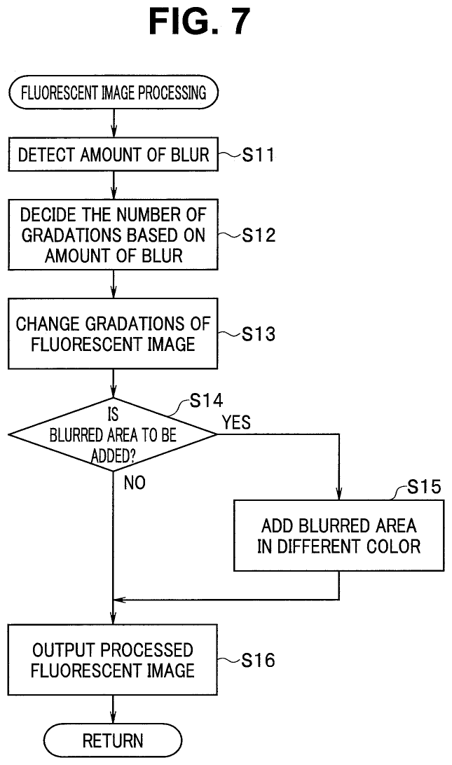

Next, FIG. 7 is a flowchart showing fluorescent image processing in the endoscope system 1.

It is assumed that focusing of a white light image has been manually performed before the process is performed as described above. Further, it is the same as the first embodiment described above that a white light image and a fluorescent image are acquired by time division.

When excitation light is emitted, and a fluorescent image is acquired, the process is started. When the fluorescent image is inputted to the fluorescent image processing portion 43, the fluorescent image processing portion 43 outputs the inputted fluorescent image to the amount-of-blur detecting portion 49.

Then, the amount-of-blur detecting portion 49 detects, for example, a contrast value of the fluorescent image. The contrast value shows that an amount of blur is small if the value is high, and the amount of blur is large if the value is low (see FIG. 11). Therefore, the amount-of-blur detecting portion 49 can detect the amount of blur of the fluorescent image according to the detected contrast value (step S11).

Note that though a contrast value is used to detect an amount of blur here, detecting the amount of blur is not limitedly performed by using a contrast value. The amount of blur may be detected by applying an appropriate amount-of-blur detection filter.

Next, the amount-of-blur detecting portion 49 outputs the detected amount of blur to the fluorescent image processing portion 43. Then, for example, as shown in FIG. 11, the fluorescent image processing portion 43 decides the number of gradations after performing the gradation conversion processing for the fluorescent image, according to the detected amount of blur (step S12).

Here, FIG. 11 is a table showing an example of causing the number of gradations after performing the gradation conversion processing according to an amount of blur of a fluorescent image to change.

In the example shown in FIG. 11, the number of gradations is set to 2 when the amount of blur is larger than a first threshold; the number of gradations is set to 3 when the amount of blur is equal to or smaller than the first threshold and is larger than a second threshold (the first threshold>the second threshold); and the number of gradations is set to 4 when the amount of blur is equal to or smaller than the second threshold.

However, FIG. 11 merely shows an example. The number of gradations after performing the gradation conversion processing may be set to a fixed value (for example, 2) irrespective of the amount of blur (in this case, the amount-of-blur detecting portion 49 may be omitted), or the number of gradations may be caused to change more finely according to the amount of blur (however, it is preferable to limit the number of gradations to an appropriate number of gradations (for example, 4 described above) because image sharpness comes close to image sharpness before performing the gradation conversion processing if the number of gradations is increased).

Thus, the fluorescent image processing portion 43 is configured to perform processing so that the number of gradations after performing the gradation conversion processing becomes small (decreases) as the amount of blur detected by the amount-of-blur detecting portion 49 becomes large (increases).

High image resolution is required from a normal image (a white light image) because it is necessary to grasp shapes of details of the subject 90. Therefore, not only causing the subject 90 to which white light is irradiated to be focused by manually adjusting the axial-direction position of the insertion portion 21 as described above but also image gradation is required. In comparison, as for a fluorescent image, since it is only required to grasp a range of the fluorescent part 91 where fluorescence is emitted, such high gradation as the gradation of a normal image (a white light image) may not be required. Therefore, the number of gradations is set small as described above to suppress decrease in sharpness.

When the number of gradations is decided as described above, the fluorescent image processing portion 43 performs the gradation conversion processing for the fluorescent image so that the decided number of gradations is obtained (step S13).

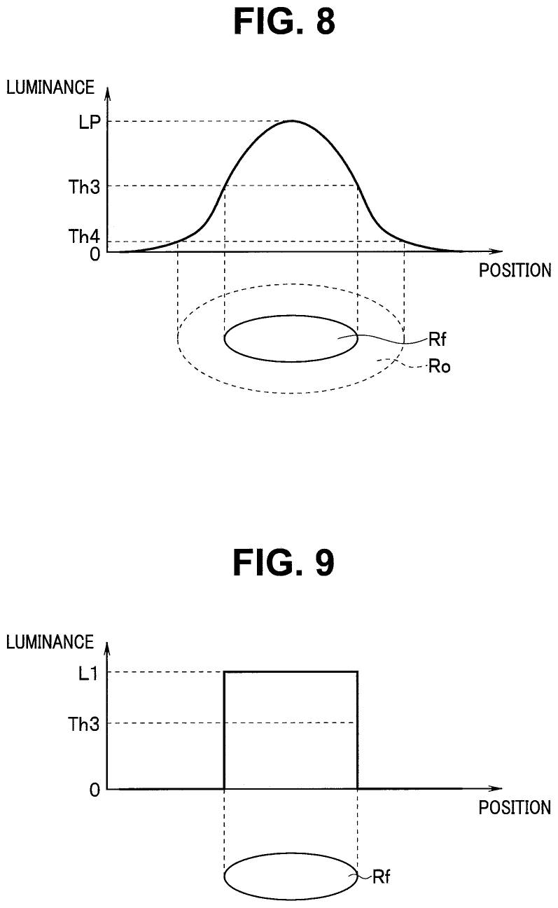

Here, FIG. 8 is a chart showing an example of spatial distribution of luminances of the fluorescent part 91 before the gradation conversion processing; and FIG. 9 is a chart showing an example of spatial distribution of luminances of the fluorescent part 91 after the gradation conversion processing of the fluorescent image is performed.

An image signal outputted from the image pickup device 16 is, for example, a signal of the number of gradations such as 10 bits (1024 gradations) or 12 bits (4096 gradations), and the fluorescent part 91 shows a smooth luminance change as shown in FIG. 8.

Then, when the number of gradations is set, for example, to 2 (that is, when binarization setting is made), such gradation conversion processing that a certain luminance value L1 is given to a pixel if a luminance value of the pixel is equal to or larger than a third threshold Th3, and a luminance value of a pixel is set to 0 if the luminance value of the pixel is smaller than the third threshold Th3 is performed.

Note that the certain luminance value L1 may be a peak luminance value LP before gradation conversion, may be decided so that an area of a part surrounded by a curved line in FIG. 8 is equal to an area of a part surrounded by a rectangle in FIG. 9 or may take another appropriate value.

When such gradation conversion processing is performed, only a fluorescent area Rf, between the fluorescent area Rf equal to or larger than the third threshold Th3 and a blurred area Ro smaller than the third threshold Th3 shown in FIG. 8, is displayed with the certain luminance value L1. Thereby, it is possible to clearly identify which part is the fluorescent area Rf, feel sharpness in the fluorescent image because the blurred area Ro does not exist, and reduce difference from the white light image in sharpness.

Next, it is judged whether the blurred area Ro around the fluorescent area Rf is to be added or not (step S14). The endoscope system 1 of the present embodiment is configured so that the user can select whether the blurred area Ro around the fluorescent area Rf is to be displayed or not, using a switch or the like of the image operating portion 4.

If it is judged at step S14 that the blurred area Ro is to be added, the fluorescent image processing portion 43 performs image processing for giving the certain luminance value L1 of color different from color of the fluorescent area Rf to pixels included in the blurred area Ro, assuming such pixels that luminance values are smaller than the third threshold Th3 and equal to or larger than a fourth threshold Th4 (the third threshold>the fourth threshold>0) as the pixels included in the blurred area Ro (step S15). Here, the fourth threshold Th4 is set to such a value that noise is not included in the blurred area Ro.

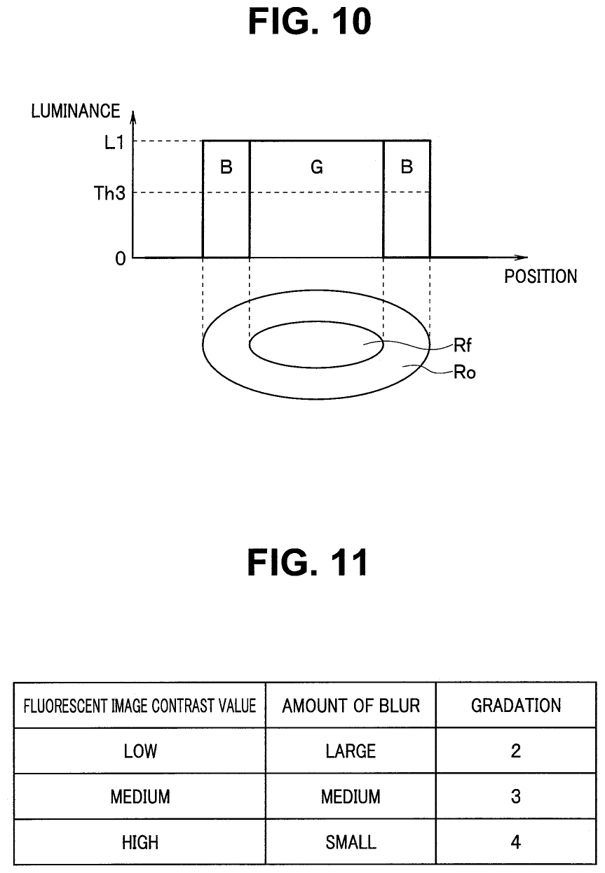

Thereby, the fluorescent part 91 is displayed by such spatial distribution as shown in FIG. 10. Here, FIG. 10 is a chart showing an example of displaying the blurred area Ro around the fluorescent area Rf in color different from the color of the fluorescent area Rf.

In the example shown in FIG. 10, the fluorescent area Rf is displayed in G (green), and the blurred area Ro is displayed in B (blue) (B given here is also an example of color that can be visually easily identified in a living body where the color component of red is relatively large). However, it goes without saying that the display color may be appropriately set.

Further, since a G component and a B component are different in rate of contribution to a luminance component (a luminance component Y is calculated, for example, as Y=0.299×R+0.587×G+0.114×B, and coefficients of G and B are different in value), the fluorescent area Rf and the blurred area Ro are not limited to being set to the same luminance value L1.

Thus, the fluorescent image processing portion 43 is configured to perform processing for the blurred area Ro for which pixel values have been converted from values equal to or larger than a predetermined threshold (the fourth threshold Th4) to 0 by the gradation conversion processing, around a fluorescent area shown by the fluorescent image signal so that the blurred area Ro is displayed in color different from the color of the fluorescent area Rf.

The fluorescent image processing portion 43 outputs to the superimposed image generating portion 44, the fluorescent image that has been gradation-conversion processed at step S13 when it is judged at step S14 that the blurred area Ro is not to be added, and outputs the fluorescent image to which the blurred area Ro has been added at step S15 when it is judged at step S14 that the blurred area Ro is to be added (step S16), and the flow returns from the fluorescent image processing to a main process not shown.

After that, the superimposed image generating portion 44 generates a superimposed image. The superimposed image generated here may be such that a white light image signal (a normal image signal) and a fluorescent image signal acquired adjoining each other in order of time division are superimposed as one image as described in the first embodiment described above or may be such that a white light image signal (a normal image signal) and a fluorescent image signal are time-division superimposed (time-division superimposition in which a white light image signal and a fluorescent image signal are alternately displayed for each frame).

Note that, because of being capable of acquiring a first image signal and a second image signal at the same focus position, the endoscope system 1 of the present embodiment is not limited to the configuration in which the first image signal and the second image signal are acquired in time division but is applicable to a configuration in which the first image signal and the second image signal are simultaneously acquired.

For example, the endoscope system 1 of the present embodiment may be applied to a simultaneous type configuration in which G light and B light are irradiated instead of white light, as reference light; excitation light which is near infrared light is also simultaneously irradiated together with the reference light; an image of the reference light is picked up by G pixels and B pixels among pixels arranged on the image pickup device 16 that is provided, for example, with a primary-color Bayer filter; and an image of fluorescence is simultaneously picked up by R pixels.

In this case, the superimposed image generating portion 44 generates the superimposed image by superimposing a reference light image signal (a normal image signal) and a fluorescent image signal acquired simultaneously (not by time division).

The gradation conversion processing for reducing gradations of a fluorescent image signal as described above may be applied to the configuration of the first embodiment (that is, the gradation conversion processing of the present embodiment is not limited to being applied to the endoscope system 1 provided with the fixed-focus imaging lens 14A).

Though automatic focus adjustment is separately performed for each of a white light image and a fluorescent image in the first embodiment described above, it is also possible to enable the endoscope system 1 to be set to such a mode that automatic focus adjustment is performed only for a white light image, and a fluorescent image is acquired at a focus position adjusted based on the white light image (that is, by omitting automatic focus adjustment for the fluorescent image) (hereinafter referred to as a single focus mode).

In other words, it is assumed that the endoscope system 1 can be set to the single focus mode in which detection of the second focused position by the focus detecting portion 46 and driving of the imaging lens 14 to the second focused position by the lens driving portion 47 are stopped.

In a case where the endoscope system 1 is set to the single focus mode, when a fluorescent image acquired by the image pickup device 16 (that is, a fluorescent image with some blur due to axial chromatic aberration) is inputted to the fluorescent image processing portion 43, the gradation conversion processing for reducing gradations of the fluorescent image signal can be performed by the fluorescent image processing portion 43.

According to the second embodiment as described above, effects almost similar to the effects of the first embodiment described above are provided, and it is possible to reduce sharpness difference (or image blur difference) that occurs on an image due to an image forming position being different when a light wavelength differs, because, in the endoscope system 1 that irradiates first light and second light with different wavelengths, gradation conversion processing for reducing gradations is performed for a second image signal acquired by irradiating the second light. Thereby, it becomes possible to suppress an uncomfortable feeling at the time of observing an image according to the first image signal and an image according to the second image signal.

Further, when the first light is assumed to be normal light (white light, reference light or the like), and the second light is assumed to be excitation light for acquiring fluorescence from a fluorescent agent, it is possible to construct the endoscope system 1 that is appropriate for fluorescent observation.

Furthermore, since the number of gradations after performing the gradation conversion processing becomes small (decreases) as an amount of blur becomes large (increases), it is possible to suppress decrease in sharpness appropriately when the amount of blur is large and give a gradation feeling of an image when the amount of blur is small.

Since it is possible to display a blurred area around a fluorescent area in color different from color of the fluorescent area, it becomes possible to clearly observe the blurred area where fluorescence occurs with a low luminance.

In addition, by providing the superimposed image generating portion 44, it is possible to appropriately reduce sharpness difference between images of a normal image signal and a fluorescent image signal that can be simultaneously observed.

In the endoscope system 1 capable of automatically adjusting a focus position in the first embodiment described above, by applying the gradation conversion processing of the present embodiment when the endoscope system 1 is set to the single focus mode, it becomes possible to reduce sharpness difference between images of a normal image signal and a fluorescent image signal without adjusting the focus position for each frame by time division.

Note that the processing of each portion described above may be performed by one or more processors configured as hardware. For example, each portion may be a processor configured with an electronic circuit or may be a circuit portion in a processor configured with an integrated circuit such as an FPGA (field programmable gate array). A processor configured with one or more CPU's may execute a function as each portion by reading and executing a processing program recorded in a recording medium.

Further, though a case where the present invention is an endoscope system has been mainly described in the above description, the present invention may be an operation method for causing the endoscope system to operate as described above, or may be a processing program for causing a computer to perform processing similar to processing of the endoscope system, a computer-readable non-transitory recording medium in which the processing program is recorded, and the like.

Furthermore, the present invention is not limited to the embodiments described above as they are, and, at an implementation stage, the components can be modified and embodied within a range not departing from the spirit of the present invention. Further, various aspects of the invention can be formed by an appropriate combination of a plurality of components disclosed in the above embodiments. For example, some components may be deleted from all the components shown in each embodiment. Furthermore, components of the different embodiments may be appropriately combined. Thus, it is, of course, possible to make various modifications and applications within the range not departing from the spirit of the invention.

What is claimed is:

1. An endoscope system comprising:
an illumination device configured to alternately irradiate first light and second light that is different from the first light in wavelength to a subject by time division;
an imaging lens configured to form an image of light from the subject as an optical image, a focus position of the imaging lens being changeable;
an imaging sensor configured to pick up the optical image of the subject to which the first light is irradiated by the illumination device to acquire a first image signal and pick up the optical image of the subject to which the second light is irradiated by the illumination device to acquire a second image signal; and
a processor; wherein
the processor detects a first focused position using the first image signal without using the second image signal and detects a second focused position using the second image signal without using the first image signal; and
the processor drives the imaging lens to the first focused position prior to acquiring the first image signal, and drives the imaging lens to the second focused position prior to acquiring the second image signal.

2. The endoscope system according to claim 1, wherein
the first light is normal light for acquiring return light from the subject, and the first image signal is a normal image signal; and
the second light is excitation light for exciting a fluorescent agent administered to the subject to acquire fluorescence from the fluorescent agent, and the second image signal is a fluorescent image signal.

3. The endoscope system according to claim 2, wherein
the processor superimposes the normal image signal and the fluorescent image signal acquired adjoining each other in order of the time division.

4. The endoscope system according to claim 2, wherein
the endoscope system is configured to be set to a single focus mode which stops the processor from detecting the second focused position and from driving the imaging lens to the second focused position; and
when the endoscope system is set to the single focus mode, the processor performs gradation conversion processing for reducing gradations of the fluorescent image signal.

5. An endoscope system comprising:
an illumination device configured to irradiate first light and second light that is different from the first light in wavelength to a subject;
an imaging lens configured to form an image of light from the subject as an optical image, a focus position of the imaging lens being fixed;
an imaging sensor configured to pick up the optical image of the subject to which the first light is irradiated by the illumination device to acquire a first image signal and pick up the optical image of the subject to which the second light is irradiated by the illumination device to acquire a second image signal; and
a processor; wherein
the processor detects an amount of blur around an area shown by the second image signal; and
the processor performs gradation conversion processing for reducing gradations of the second image signal so that the number of gradations after performing the gradation conversion processing becomes smaller as the detected amount of blur becomes larger.

6. The endoscope system according to claim 5, wherein
the first light is normal light for acquiring return light from the subject, and the first image signal is a normal image signal; and
the second light is excitation light for exciting a fluorescent agent administered to the subject to acquire fluorescence from the fluorescent agent, and the second image signal is a fluorescent image signal.

7. The endoscope system according to claim 6, wherein
the processor performs processing for a blurred area for which pixel values have been converted from values equal to or larger than a predetermined threshold to 0 by the gradation conversion processing, around a fluorescent area shown by the fluorescent image signal so that the blurred area is displayed in color different from color of the fluorescent area.

8. The endoscope system according to claim 6, wherein
the processor superimposes the normal image signal and the fluorescent image signal.
